(19) United States
(12) Patent Application Publication
(10) Pub. No.: US 2007/0038118 A1
(43) Pub. Date: Feb. 15, 2007

(54) Subcutaneous tissue imager

(76) Inventors: Marshall Thomas DePue, San Jose, CA (US); Tong Xie, San Jose, CA (US)

Correspondence Address: AVAGO TECHNOLOGIES, LTD., P.O. BOX 1920, DENVER, CO, 80201-1920, US

(21) Appl. No.: 11/200,631

(22) Filed: Aug. 10, 2005

(51) Int. Cl. A61B 6/00

(52) U.S. Cl. 600/476

(57) ABSTRACT

An imaging device comprises a light source configured to illuminate a subcutaneous tissue structure with substantially coherent light, a sensor module, an imaging lens interposed between the subcutaneous tissue structure and the sensor module to focus an image of the subcutaneous tissue structure at the sensor module.

### BACKGROUND

Every human body has generally the same make-up of a musculoskeletal system, internal organs, skin, and more. However, despite a world population over six billion, nearly every person has a unique variation of those components. This phenomenon is particularly true of the circulatory system including the heart, arteries, and veins. For example, while a blood vessel may be positioned in generally the same area from person-to-person, its exact position and interconnection to other proximate blood vessels, and any branching from those blood vessels, is distinctly different in each person.

This anatomical wonder has several implications. In one implication, finding the exact location of a vein for inserting a medical instrument, such as a catheter or syringe needle can be challenging. This search is particularly challenging for a nurse or physician when the patient has an abundance of subcutaneous fatty tissue, thereby rendering the underlying vessels difficult to detect by finger palpation. Patients that are severely dehydrated or elderly pose related challenges as their veins tend to retract into surrounding tissue and the overall size of the vein shrinks, making them difficult to find.

One conventional approach at dealing with this situation includes illuminating the skin over an area of interest to produce video images of vessels underlying the skin, and then projecting those video images back onto surface of the skin as a virtual map of the underlying vessels. Unfortunately, these systems are relatively large and bulky, being cumbersome for use in a clinic or outpatient setting. Moreover, using conventional light emitting diodes (LEDs) to illuminate skin tissue for imaging purposes, as typically arranged with multiple filters and/or polarizers, produces a relatively coarse image contrast. These relatively coarse images hinder accurate determination of the underlying vessel structure.

The anatomical diversity from person-to-person also enables individuals to be uniquely identified relative to each other, such as by comparison of fingerprints. However, even this time-honored identification method is not foolproof as sophisticated criminals have developed ways to undermine the accuracy of fingerprint identification. Accordingly, additional biometric features, such as retinal identification and voice recognition tools, are being developed to assist in uniquely identifying a person.

Accordingly, the variations of human anatomy from person-to-person continue to present challenges in reliably identifying the exact features of anatomy for a particular person, as well as challenges in how to perform sophisticated tasks based on images of those anatomical features.

### SUMMARY

Embodiments of the invention are directed to a subcutaneous imaging device. In one embodiment, an imaging device comprises a light source, a sensor module, and an imaging lens. The light source is configured to illuminate the subcutaneous tissue structure with a substantially coherent light. The imaging lens is interposed between the subcutaneous tissue structure and the sensor module to focus an image of the subcutaneous tissue structure at the sensor module.

### BRIEF DESCRIPTION OF THE DRAWINGS

FIG. 1 is block diagram illustrating an imaging device, according to an embodiment of the invention.

FIG. 2A illustrates a displayed digital representation of a subcutaneous tissue structure, according to an embodiment of the present invention.

FIG. 2B illustrates a reconstructed image of a subcutaneous tissue structure based on the digital representation of FIG. 2A, according to an embodiment of the present invention.

FIG. 3 is a flow diagram illustrating a method of imaging subcutaneous tissue structure, according to an embodiment of the invention.

FIG. 4 is a diagram illustrating a person identification device, according to an embodiment of the present invention.

FIG. 5 is a diagram illustrating a guide mechanism of an identification device, according to an embodiment of the present invention.

FIG. 6 is a diagram illustrating a perspective view of a person identification device, according to an embodiment of the present invention.

FIG. 7 is a diagram illustrating a medical instrument insertion device, according to an embodiment of the present invention.

FIG. 8 is a diagram illustrating application of a medical instrument insertion device, according to an embodiment of the present invention.

FIG. 9 is diagram illustrating a medical instrument insertion device, according to an embodiment of the present invention.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following Detailed Description, therefore, is not to be taken in a limiting sense, and the scope of the present invention is defined by the appended claims.

Embodiments of the invention are directed to devices for producing images of a subcutaneous tissue structure of a body portion. In particular, embodiments of the invention illuminate a subcutaneous tissue structure with substantially coherent light to enable sensing an image of the illuminated subcutaneous tissue structure at a sensor module via an imaging lens. The imaging lens is positioned, shaped, and sized to focus the image at the sensor module from a location beneath a body surface at the subcutaneous tissue structure.

In one aspect, these components of the imaging device are contained within a single housing. In another aspect, the imaging device is portable.

In one embodiment, a person identification device obtains an image of a subcutaneous tissue structure, such as a pattern of a capillary vein structure in a finger, to uniquely identify an individual. In one aspect, the image is stored in a database for future reference. This image acts as a “vein print” to uniquely identify that individual apart from all other individuals. In one aspect, the image is compared to at least one record image from a database of stored images of subcutaneous tissue structures, with each record image corresponding to a unique individual.

In one embodiment, the image is used to confirm that the identity of an individual seeking access to a building, room, equipment, computer, etc. matches at least one record image corresponding to an authorized individual (one having authority to gain access). In another embodiment, the image is used to determine whether the identity of the individual matches an identity of a plurality of persons among a group, such as wanted criminals.

In another embodiment, the “vein print” image is used along with conventional analog or digital fingerprint methods, or other biometric identifiers (e.g. retinal detection, voice recognition, etc.), to enable the contemporaneous application of more than one identification method.

In another embodiment, a medical instrument insertion device obtains an image of a subcutaneous tissue structure, such as a vein in a forearm, to identify a target vein suited for insertion of a medical instrument, such as a catheter or syringe needle. In one aspect, the image of a target vein is compared against a threshold for a size and/or shape of a suitable vein to enable an indication to the operator whether the target vein should be used. In one aspect, the image of the target vein is displayed to enable visualization of the target vein before, during, or after insertion of a medical instrument into the vein. In one embodiment, the imaging device is removably coupled to the medical instrument while, in other embodiments, the imaging device is permanently coupled to a portion of the medical instrument. In another embodiment, the imaging device is not coupled to the medical instrument.

Embodiments of the invention use a light source that produces substantially coherent light to illuminate a subcutaneous tissue structure. In one embodiment, the substantially coherent light is an infrared light. An imaging lens has a size, shape, and position to focus scattered and reflected light from the illuminated subcutaneous tissue structure beneath the skin (or body surface) as an image at a sensor module. The sensor module produces a digital representation of the subcutaneous tissue structure. This digital representation is stored and/or displayed, with displayed images being in a grid of pixel values or as a reconstructed picture of the subcutaneous tissue structure.

In one embodiment, the body portion to which the subcutaneous imaging device is applied comprises a human body portion. In another embodiment, the body portion comprises an animal body portion.

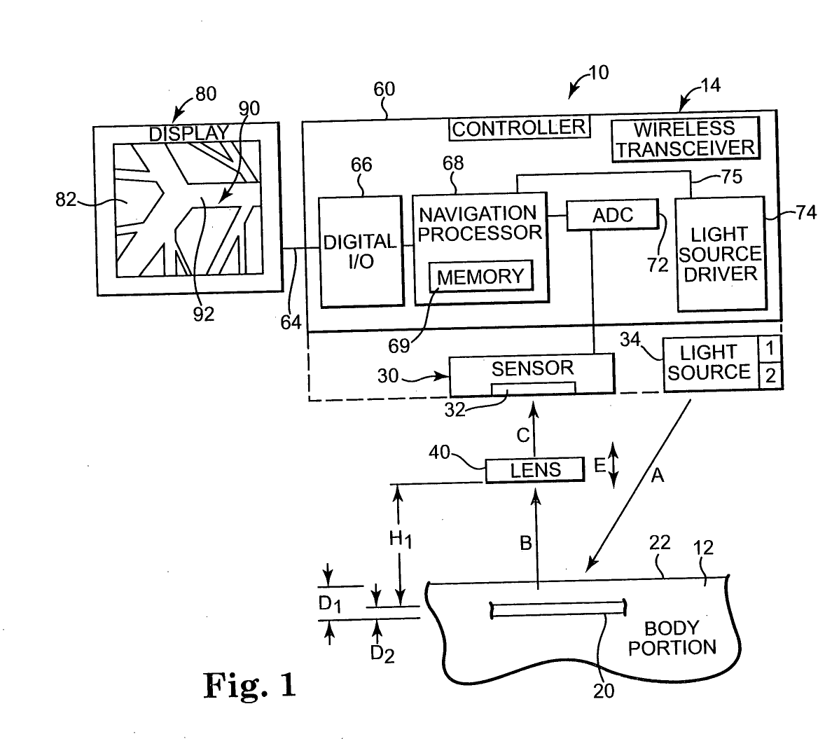

Examples of a subcutaneous imaging device according to embodiments of the invention are described and illustrated in association with FIGS. 1-9.

FIG. 1 is a block diagram illustrating major components of an imaging device 10, according to one embodiment of the invention. As shown in FIG. 1, imaging device 10 obtains an image of a subcutaneous tissue structure 20 below surface 22 of body portion 12. In one aspect, surface 22 is a skin surface of a body portion, such as a forearm, fingertip, leg, torso, etc.

In another aspect, surface 22 is a non-skin body surface such as a surface of an internal organ, conduit, or other internal body portion. In this regard, the subcutaneous tissue structure is considered a subsurface tissue structure because surface 22 is not strictly a skin surface but a surface of another organ or other body portion.

In one embodiment, imaging device 10 comprises sensor module 30, light source 34, imaging lens 40, and display 80. In operation, according to one embodiment, light source 34 emits light (A) through body portion 20 to illuminate subcutaneous tissue structure 20 and thereby produce scattered optical effects caused by varying absorptive and reflective properties of subcutaneous tissue structure 20. By a well known mechanism, light in the infrared spectrum that is directed at blood-carrying vessels, such as a vein, tends to be absorbed while light directed at other tissues, such as fatty tissues, etc. tends to be reflected and scattered away from those tissues.

Imaging lens 40 acts to focus light that is reflected and/or scattered at subcutaneous tissue structure onto sensor array 32 of sensor module 30. Imaging lens 40 is positioned and directed to be generally perpendicular to surface 22 and subcutaneous tissue structure 20. Light source 34 is generally positioned so that light from light source 34 travels along a path (A) at an angle to surface 22 to enhance scattering, absorption, etc. of light to highlight spatial differences between features of subcutaneous tissue structure 20.

In one aspect, imaging lens 40 is sized and shaped have a focal length corresponding to a depth of subcutaneous tissue structure 20 beneath skin surface 22. In another aspect, imaging lens 40 has a fixed position relative to sensor array 32 and skin surface 22 to enable application of the focal length of the imaging lens at subcutaneous tissue structure 20. In one aspect, imaging lens 40 is selectively movable (as represented by directional arrow D) relative to the sensor module 30 and/or subcutaneous tissue structure 20 for adjusting the depth of focus beneath surface 22. In one aspect, imaging lens 40 is positioned so that its optical axis is generally perpendicular relative to skin surface 22 and therefore relative to subcutaneous tissue structure 20.

Optical effects reflected from subcutaneous tissue structure 20, in response to illumination from light source 34, are received at sensor array 32 (e.g., a photodetector array) and processed to form a digital representation of the imaged tissue structure 20, as described below in further detail.

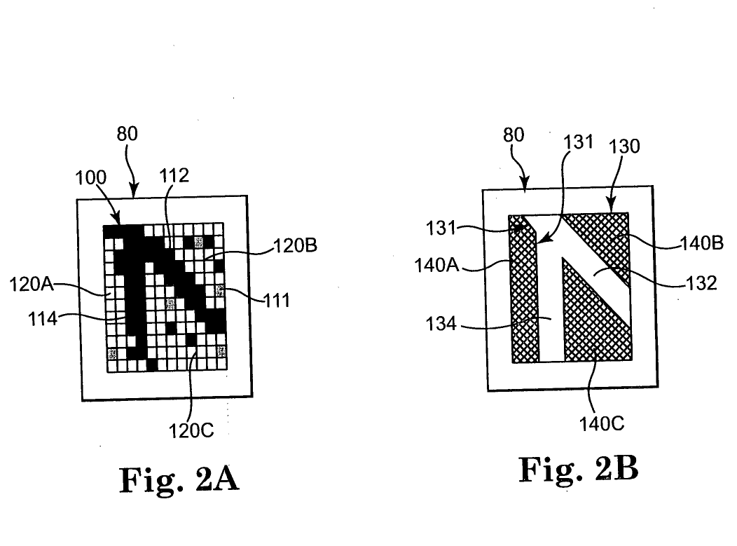

In one embodiment, display 80 comprises a screen for displaying a reconstructed image 90 of a subcutaneous tissue structure 20 including vessel structure 92 and other tissue 82, such as fatty tissue. In one embodiment, imaging device 10 omits display 80. Instead, other types of indicators, such as auditory tones or light indicators, are used to indicate information about features of the subcutaneous tissue structure 20. These features, and additional features of a display associated with an imaging device 10, according to an embodiment of the invention, are illustrated and described in association with FIGS. 2A-9.

In one embodiment, sensor module 30 and light source 34 form a portion of optical imaging sensor integrated circuit (IC) 60. As shown in FIG. 1, optical navigation sensor 60 includes digital input/output circuitry 66, navigation processor 68, analog to digital converter (ADC) 72, sensor array (or photodetector array) 34 of sensor module 30, and light source driver circuit 74. In one embodiment, sensor array 32 comprises a CMOS photo detector array.

In one embodiment, each photodetector in sensor array 32 provides a signal that varies in magnitude based upon the intensity of light incident on the photodetector. The signals from sensor array 32 are output to analog to digital converter (ADC) 72, which converts the signals into digital values of a suitable resolution. The digital values represent a digital image or digital representation of the illuminated portion of subcutaneous tissue structure 20. The digital values generated by analog to digital converter (ADC) 72 are output to image processor 68. The digital values received by image processor 68 are stored as a frame within memory 69. In one embodiment, the digital image or digital representation of the illuminated portion of subcutaneous tissue structure 20 is displayed on display 80. In one embodiment, memory 69 comprises a database for storing an array of images of subcutaneous tissue structures, with each image in the array corresponding to a different individual or a different body portion. Use of database 68 is described further in association with FIGS. 2-9.

Various functions performed by sensor module 30 and navigation sensor circuit 60 (FIG. 1) may be implemented in hardware, software, firmware, or any combination thereof. The implementation may be via a microprocessor, programmable logic device, or state machine. Components of the present invention may reside in software on one or more computer-readable mediums. The term computer-readable medium as used herein is defined to include any kind of memory, volatile or non-volatile, such as floppy disks, hard disks, CD-ROMs, flash memory, read-only memory (ROM), and random access memory

In one embodiment, light source 34 is a substantially coherent light source. In one embodiment, light source 34 is a laser. In one form of the invention, light source 24 is a vertical cavity surface emitting laser (VCSEL) diode. In another form of the invention, light source 34 is an edge emitting laser diode. In one aspect, light source 34 is polarized. Both the polarized nature and the substantially coherency of the light of light source 34 produces high contrast images that are especially suitable for imaging subcutaneous tissue structures, thereby obviating additional structures such as conventional polarizing optics and conventional filters that are generally associated with the use of generally incoherent light sources (e.g. a conventional LED having a broadband wavelength) in imaging applications. In another embodiment, light source 34 comprises a combination of a broadband light source (e.g., a LED producing generally incoherent light) coupled with a light conditioner adapted to produce substantially coherent light from the broadband light source.

Light source 34 is controlled by driver circuit 74, which is controlled by navigation processor 68 via control line 75. In one embodiment, control line 75 is used by navigation processor 68 to cause driver circuit 74 to be powered on and off, and correspondingly cause light source 34 to be powered on and off.

In one embodiment, light source 34 comprises a light source producing light in a single wavelength in a range known to those skilled in the art that is suitable for scattering and absorption by subcutaneous tissue structures. In one aspect, a first wavelength of light used is within a range of about 830 nanometers to 850 nanometers. In another aspect, light source 34 comprises a light source producing light of two different wavelengths (as represented by identifiers 1 and 2 of light source 34 in FIG. 1), with each wavelength selected to fall within the range known to those skilled in the art that to be suitable for scattering and absorption by subcutaneous tissue structures. In one aspect, the light absorption coefficient for a blood vessel varies as a function of wavelength of the light.

Illuminating a vein structure with two different wavelengths of light produces an image corresponding to a three dimensional representation of the subcutaneous tissue structure, thereby providing information about the relative depth of adjacent vein structures. In one aspect, a varying wavelength of light is provided by configuring light source 34 as a tunable laser light source that enables varying the wavelength of light emitted at the selection of a user. In another aspect, light source 34 comprises a light source die including multiple laser light sources, each producing a different wavelength of light.

In another aspect, detecting a depth dependent structure of several veins of the subcutaneous tissue structure 20 enables confirmation that the object being imaged corresponds to a live human being or live animal. This feature foils attempts to use inanimate decoys, such as fake fingers or optical patterns, that are intended to trick imaging device 10 into making a positive identification of a subcutaneous tissue structure.

Moreover, the absorption coefficient of light is based, in part, on the amount of oxygen in the vessel structure. Accordingly, as oxygen is reduced or increased within the target vein structure, a corresponding change will occur in the image of the illuminated target vein structure. In one aspect, a condition of the body, such as stress, is inferred based on the absorption coefficient over time to enable operation of the imaging device as a light detector or stress meter.

In another aspect, detecting that a vein of the subcutaneous tissue structure 20 carries oxygen enables additional confirmation that the object being imaged corresponds to a live human being or live animal. This feature foils attempts to use inanimate decoys, such as fake fingers or optical patterns, that are intended to trick imaging device 10 into making a positive identification of a subcutaneous tissue structure.

Finally, imaging device 10 comprises wireless transceiver 78 that is configured for short range wireless communication with a video monitor or remote station to enable display and/or evaluation of the images of the subcutaneous tissue structure.

FIG. 2A illustrates one example of a digital representation of an image of a subcutaneous tissue structure, according to an embodiment of the present invention and FIG. 2 B illustrates an example of a reconstructed image of the of subcutaneous tissue structure based on the digital representation of that tissue structure in FIG. 2A.

As shown in FIG. 2A, display 80 displays a frame 100 that shows a digital representation of subcutaneous tissue structure 20 (FIG. 1) on a pixel-by-pixel basis in which different shading levels (e.g. white, black, gray, etc.) represent different structural features (e.g., veins, fat, etc.) associated with the subcutaneous tissue structure 20. In one embodiment, frame 100 comprises an array of pixels including a region 114 of dark pixels (e.g. black pixels) and region(s) 120A,120B, 120C of light pixels (e.g. white pixels). In addition, some pixels 111 comprise gray pixels, which have an intermediate intensity level. The relative darkness (i.e. gray-level intensity) of a pixel corresponds generally to the relative absorption of light caused by various anatomical features of the subcutaneous tissue structure.

In one aspect, darker pixels generally correspond to a blood carrying vessel and lighter pixels generally correspond to other tissues, such as fatty tissues. In some instances, the lightness or darkness of a pixel corresponds to whether a given feature is within or outside the depth of focus of the imaging device relative to the subcutaneous tissue structure. In another aspect, image processor 68 categories each pixel as either a blood-carrying pixel or a non-blood pixel. In another aspect, other subcutaneous structures are represented by intermediate level pixels.

FIG. 2B illustrates a reconstructed image of subcutaneous tissue structure, according to an embodiment of the invention, represented by viewable frame 130. Frame 130 is constructed by processing the digital representation of frame 100 to produce an analog-type image to provide a more picture-like representation of the subcutaneous tissue structure 20.

As shown in FIG. 2B, frame 130 comprises display vein structure 131 including first vessel 132, second vessel 134 and surrounding non-vessel structures 140A, 140B, 140C. Each portion of reconstructed frame 130 generally corresponds, on a pixel-by-pixel basis, to each gray-level portion of digital representation frame 100. Display vein structure 131 enables a viewer to recognize familiar physiological structures consistent with direct observation (unaided or microscopically aided) of the subcutaneous tissue structure 20.

FIG. 3 illustrates a method 200 of identifying subcutaneous tissue structures using an imaging device, according to an embodiment of the invention. As shown in FIG. 3, at 202, a subcutaneous tissue structure is illuminated with substantially coherent light from a light source. In one aspect, the light source produces the substantially coherent light from a laser light source.

At 204, an image of the illuminated subcutaneous tissue structure is sensed at sensor module through an imaging lens focused beneath a body surface at a target subcutaneous tissue structure. At 205, the image is stored within a memory. In one aspect, the memory comprises a database of stored images of subcutaneous tissue structures from one or more individuals.

In one embodiment, at 206, the stored image is compared with at least one record image stored in a database to identify an individual. In one aspect, the at least one record image comprises a plurality of record images with each record image corresponding to a different unique individual.

In another embodiment, at 208, the image is displayed to facilitate insertion of an instrument though a body surface into the subcutaneous tissue structure of a body portion. In one aspect, the imaging device is coupled in close proximity to the instrument to facilitate obtaining the image of subcutaneous tissue structure adjacent the position of the instrument (which is external to the surface of the body portion containing the subcutaneous tissue structure). The imaging device and instrument are discarded after use. In another aspect, the imaging device is removably coupled relative to the instrument, so that the imaging device is separable from the instrument for later re-use with another instrument.

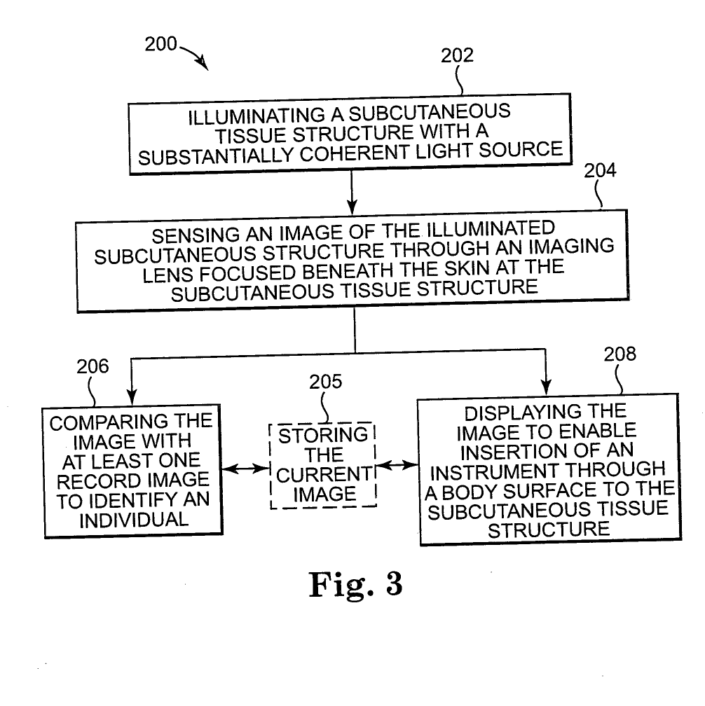

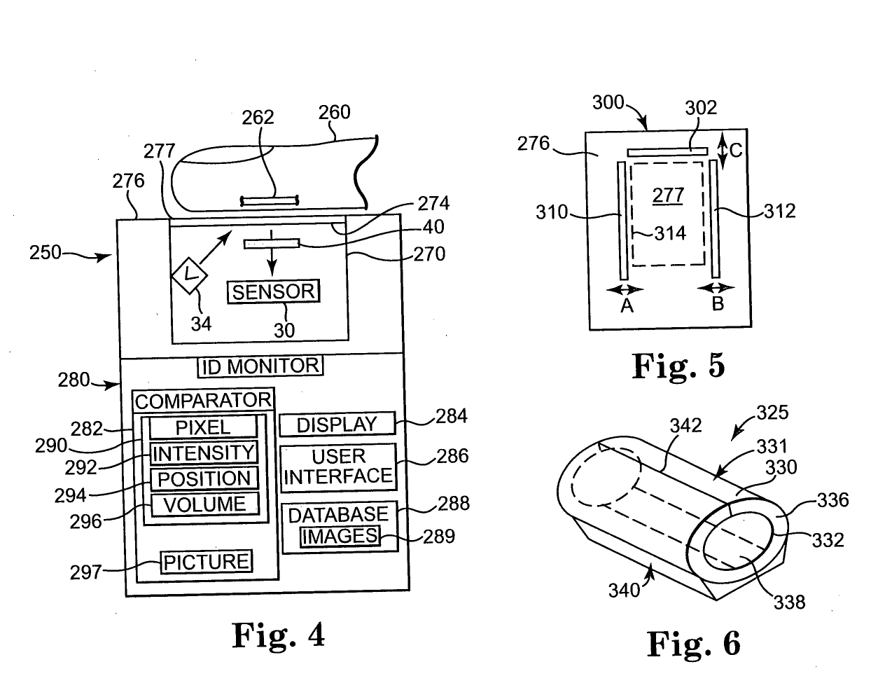

In one embodiment, method 200 is performed using imaging device 10 as previously described and illustrated in association with FIG. 1, and as well as any one of imaging devices 250, 325, 400 and/or 600 as will be described in association with FIGS. 4-9.

FIG. 4 is a perspective view illustrating an imaging device 250, according to an embodiment of the invention. Imaging device 250 comprises a person identification device that enables uniquely identifying individuals by a subcutaneous tissue structure 262 of a body portion, such as a finger tip 260, as shown in FIG. 4. In one embodiment, imaging device 250 comprises substantially the same features and attributes as imaging device 10, as previously described in association with FIG. 1, as well as additional features described and illustrated in association with FIGS. 4-6.

As shown in FIG. 4, imaging device 250 comprises imaging mechanism 270, including light source 34, sensor module 30, transparent window 274 having a contact surface 277 for receiving placement of finger tip 260. A housing 276 provides a fixed station for supporting and containing imaging mechanism 270. Imaging mechanism 270 obtains an image of subcutaneous tissue structure 262 with light source 34, lens 40 and sensor module 30 operating in substantially the same manner as previously described for imaging device 10 (shown in FIG. 1), except that light traveling from light source 34 to sensor module 30 passes through transparent window 274 of housing 276. In one aspect, transparent window 274 is replaced by a contact surface 277 having an opening to enable light from light source 34 and to sensor module 30 to pass in and out of imaging mechanism 270 relative to finger tip 260.

In another aspect, contact surface 277 (FIGS. 4-5) includes a removable, transparent film (or sheet) that is discarded after each use to insure that artifacts (e.g., grease, dirt, scratches, etc.) do not interfere with optical imaging performed by imaging device 10, 250.

In one embodiment, imaging device 270 also comprises an identification (ID) monitor 280 to enable the use of images produced by imaging mechanism 270 to identify an individual. As shown in FIG. 4, imaging device 270 comprises comparator 282, display 284, user interface 286, and database 288. Comparator 282 comprises pixel module 290 including intensity parameter 292, position parameter 294, and volume parameter 296. Database 288 comprises a memory and stores an array of record images 289, as well as storing a current image.

Comparator 282 enables comparison of a current stored image with stored record images 289 in database 288. Record images 289 comprise previously stored images of one or more individuals.

Pixel module 290 of comparator 282 enables selecting how different features of an image of a subcutaneous tissue structure will be compared. For example, pixels of one image are compared with corresponding pixels of another image according to any one or more of an intensity (via intensity parameter 292), a position (via position parameter 294), and a volume (via volume parameter 296) of the pixels.

In one aspect, volume parameter 296 controls the volume of pixels of the current image that must substantially match a corresponding pixels of a stored record image (according to intensity and/or position) in order to consider the current image to match a stored image. The volume of “matching” pixels is set via volume parameter 296 by a number of matching pixels, a percentage of matching pixels, or other mechanisms for expressing a volume of matching pixels that match between a stored current image and a stored record image.

In one aspect, intensity parameter 292 controls a threshold of the light-dark intensity of a single pixel, a group of pixels, or a pixel region of an image frame used to determine whether a pixel in a current image substantially matches a corresponding pixel in a stored record image. In another aspect, position parameter 294 controls a threshold of position-matching for a single pixel, a group of pixels, or a pixel region of an image frame to determine whether a position of a pixel (or group of pixels, or pixel region) in a current image substantially matches the position of a corresponding pixel (or group of pixels, or pixel region) in a stored record image.

Picture module 297 of comparator 282 enables displaying a picture of an image of a subcutaneous tissue structure so that a user can make their own visual comparison of a current image relative to a stored image. For example, a picture-type frame (substantially the same as frame 130 in FIG. 2B) of a current image is compared to a picture-type frame of one or more stored record images.

User interface 286 enables controlling the previously described parameters of comparator 282, features of display 80 (such as, on/off function, enlarge function, etc.), and functions of imaging mechanism 270 (e.g., position of lens 40, on/off, etc.).

FIG. 5 is a top plan view of positioning mechanism 300, according to an embodiment of the invention, for use with imaging device 250. As shown in FIG. 5, positioning mechanism 300 comprises first guide 302, second guide 310, and third guide 312, as well as marked boundary 314 of contact surface 277. In one embodiment, positioning mechanism 300 is disposed in close proximity to contact surface 277 on a top portion of housing 276. Marked boundary 314 provides a target over which the finger tip is to be placed. Guides 302, 310, and 312 are arranged to enable a fingertip 260 to be correctly positioned over contact surface 277 to increase the likelihood of an image being obtained via imaging mechanism 270 that is consistent with the positioning of a fingertip of the same individual or of other individuals. Accordingly, this positioning mechanism 300 enhances the ability to compare images based on similarly positioned fingertips.

In one aspect, positioning mechanism 300 comprises clips, straps, conformable flaps, etc to help maintain position of fingertip 260 relative to contact surface 277.

FIG. 6 is a perspective view of an imaging device 325, according to an embodiment of the invention. As shown in FIG. 6, imaging device 325 comprises a finger guide 331 and an imaging mechanism 340. Finger guide 331 enables removably inserting a finger tip within finger guide 331 to position a finger tip relative to imaging mechanism 340 for obtaining an image of a subcutaneous tissue structure 262 of the finger tip 260.

In one embodiment, imaging device 325 is portable. In another embodiment, imaging device 325 comprises a portion of stationary imaging system.

In one embodiment, imaging device 325 comprises substantially the same features and attributes as imaging devices 10 as previously described in association with FIGS. 1-3. In one aspect, imaging mechanism 340 comprises substantially the same features and attributes as imaging device 250 as previously described in association with FIG. 4, particularly including imaging mechanism 270 and/or ID monitor 280, in which contact surface 338 of FIG. 6 provides substantially the same features and attributes as contact surface 277 (FIG. 4-5).

As shown in FIG. 6, finger guide 331 comprises generally tubular member 330 including opening 332 as defined by side wall 336. In one aspect, tubular member 330 is made of a resilient, flexible material to adapt to different sized finger tips. In one aspect, tubular member 330 comprises a seam 342 enabling side walls 336 to be folded open and away from each other to facilitate insertion of the finger tip 260 within finger guide 331, wherein the resiliency of tubular member 330 enables the side walls 336 to return to a closed position about the finger tip 260 after the finger tip 260 is in place relative to contact surface 338. In another embodiment, tubular member 330 is made from a semi-rigid material and side walls 336 of finger guide 330 operate as opposed members of a clip that are openable along separation seam 342.

In one aspect, imaging mechanism 340 includes its own power source and memory. In another aspect, imaging mechanism 340 additionally comprises a wireless transceiver (as in imaging device 10) for wirelessly transmitting an image obtained by imaging device 325 to a remote manager for comparison of that current image with a database of images. In one example, portable imaging device 325 is used by security personnel or law enforcement personnel to obtain a digital “vein print” of an individual for immediate or later comparison with a database of “vein prints”. In one example, the “vein print” is not compared with other “vein prints”, but merely obtained and stored for future use, as would a conventional fingerprint.

Accordingly, a subcutaneous imaging device is used to map a “vein print” of an individual for uniquely identifying that individual relative to other individuals.

FIG. 7 illustrates an imaging system 400, according to an embodiment of the invention. As shown in FIG. 7, system 400 comprises a portable device 401 comprising imaging device 402 and instrument 404 connected together via coupler 406. In one embodiment, imaging device 402 comprises indicator mechanism 420 and/or image manager 450.

In one embodiment, imaging device 402 comprises substantially the same features and attributes as imaging devices 10, 250 as previously described in association with FIGS. 1-4, including sensor 30, light source 34, and imaging lens 40 (not shown in FIG. 7). In one aspect, imaging device 402 comprises substantially the same features and attributes as imaging device 250 as previously described in association with FIG. 4, particularly including imaging mechanism 270 and/or ID monitor 280. However, in one embodiment, imaging device 402 does not contact surface 476 of body portion 474 when obtaining an image of subcutaneous tissue structure 478 while in other embodiments, such contact is enabled when the image is obtained.

Instrument 404 includes a penetrator 405 (e.g., needle, catheter tip) for insertion into and through a surface 476 (e.g., skin, organ side wall, etc.) of body portion 474 for ultimate insertion of penetrator 405 into a blood carrying vessel 478, such as a vein. In one embodiment, instrument 404 comprises a catheter such as a heart catheter (e.g. angioplasty catheter, angiogram catheter, introducer sheath, etc.) while in other embodiments, instrument 404 comprises a syringe needle or stylet. Imaging device 402 is maintained in close proximity to instrument 404 via coupler 406 to enable obtaining an image of subcutaneous tissue structure 477 to identify blood carrying vessel 478, thereby insuring that penetrator 405 hits its target vein (or other body vessel) upon insertion.

Images of subcutaneous tissue structure 477 are used to indicate that an appropriate vessel 478 has been found. As shown in FIG. 7, in one embodiment, system 400 comprises indicator 420. Indicator 420 comprises one or more of a light indicator 422, a display indicator 424, and an auditory indicator 426. Light indicator 422 enables a light to flash or to shine to indicate the presence of a suitable vessel 478. Auditory indicator 426 enables an auditory tone (intermittent or constant) to indicate the presence of a suitable vessel 478. The light indications or auditory tones, respectively, cease when the imaging device 402 is no longer positioned over a suitable vessel, thereby indicating, by virtue of coupling between imaging device 402 and instrument 404, that penetrator 205 of instrument 404 is not positioned for insertion into a suitable vessel 478.

In one aspect, in a manner substantially the same as display 80 in FIG. 1, display 424 displays a visual picture of the subcutaneous tissue structure 477. The visual picture reveals whether or not imaging device 402 and instrument penetrator 405 are positioned over a suitable vessel 478 within subcutaneous tissue structure 477.

In one aspect, image manager 450 acts in cooperation with indicator 420 in which image processing and correlation algorithms are used to compare continually acquired current images of a vein structure against a model image of vein. A substantial match between one of the current images and the model image triggers indicator mechanism 420 to produce a light or auditor cue that a target vein has been identified.

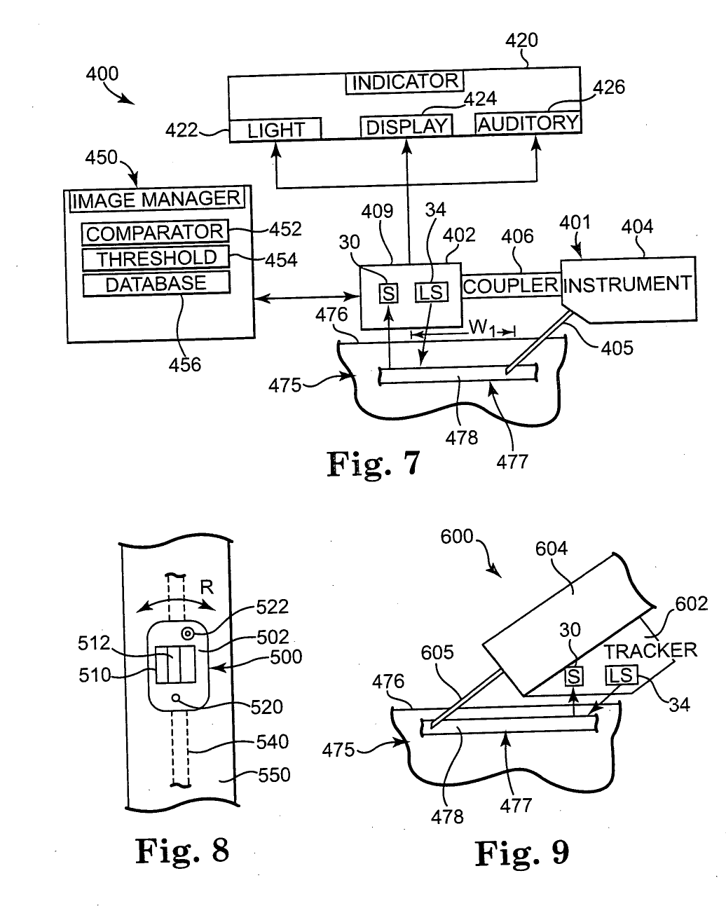

In one aspect, one or more components of indicator 420 are incorporated into housing 409 of imaging device 402, as further illustrated in FIG. 8.

In one embodiment, imaging device 402 comprises image manager 450. As shown in FIG. 7, image manager 450 comprises comparator 452, threshold parameter 454, and database 456. In one embodiment, comparator 452 comprises substantially the same features and attributes as comparator 282 of imaging device 250, as previously described in association with FIG. 4. Accordingly, comparator 452 compares current images of a subcutaneous tissue structure 477 against predetermined criteria, such as stored images of a model vessel or of an actual vessel having a size, shape or position deemed suitable for insertion of penetrator 405. In one aspect, a contrast ratio of a vein acts as a parameter for evaluation relative to a model contrast ratio of the predetermined criteria. In one aspect, threshold parameter 454 enables an operator or designer to determine the threshold size, shape, and/or position of a vessel deemed suitable for insertion of penetrator 405.

In one aspect, database 456 comprises a memory and stores images of suitable vessels according to different portions of the body (e.g., finger, arm, leg, torso, etc.).

In one embodiment, a wireless transceiver 78 (FIG. 1) in imaging device 402 enables transmission of real time images to a remote video monitor to enable visualization of a subcutaneous tissue structure in relation to instrument 404 during a procedure to facilitate operation of instrument relative to subcutaneous tissue structure.

In one embodiment, database 456 comprises a memory and stores images taken via imaging device 402 during operation of instrument 404 relative to subcutaneous tissue structure to document the procedure for insurance purposes, legal purposes, medical records, research documentation, etc.

In one embodiment, coupler 406 permanently secures imaging device 402 relative to instrument 404. In this case, imaging device 402 would most likely be discarded after use with the used instrument 404. In another embodiment, coupler 406 removably secures imaging device 402 relative to instrument 404 so that after use of instrument 404, imaging device 402 is separable from instrument 404 for later re-use with a different instrument 404 of the same type or different type.

In addition, in another embodiment, imaging device 402 is separate from (or separable from) and independent of instrument 404 to enable its use as diagnostic tool to evaluate a subcutaneous tissue structure on various parts of the body using images of the subcutaneous tissue structures.

In one embodiment, coupler 406 is rigid and has a fixed length. In another embodiment, coupler 406 is resiliently flexible and has a variable length.

FIG. 8 illustrates imaging device 500 in use, according to an embodiment of the invention. In one embodiment, imaging device 500 comprises substantially the same features and attributes as imaging device 402. Imaging device 502 comprises housing 502 which supports display 510, light module 520, and/or audio module 522, which have substantially the same features and attributes as display 424, light indicator 422, and auditory indicator 426 of FIG. 7. Display 510 shows an image 512 of vessel 478 that is positioned underneath imaging device 502. FIG. 8 also shows a rotational path R representing lateral rotation of device 502 during an attempt to find vessel 478 via imaging device 502.

FIG. 9 illustrates system 600, according to an embodiment of the invention. As shown in FIG. 9, system 600 comprises instrument 604 with penetrator 605 and imaging device 602, which have substantially the same features as instrument 404 and imaging device 402 except for imaging device 602 being secured underneath instrument 608 relative to a body 608 of instrument 604. This arrangement positions imaging device 602 on a side of penetrator 605 generally opposite to the position of imaging device 402 relative to penetrator 405 shown in FIG. 7, thereby demonstrating that the imaging device is not limited to the particular position shown in FIG. 7. In some instances, it is desirable to have imaging device in front of instrument (as in FIG. 7) or in back of instrument (as in FIG. 9).

Embodiments of the invention enable accurate imaging of subcutaneous tissue structures, such as blood vessels, for either identifying an individual uniquely apart from other individuals or identifying a target blood vessel to perform a medical procedure at that target blood vessel. Use of substantially coherent light source(s) to illuminate the subcutaneous tissue structure eliminates the use of multiple filters and/or polarizers found in conventional tissue imaging devices that use incoherent illumination. Finally, the small scale of the components (e.g. light source, sensor module, and lens) enables a small form factor akin to a pocket-size imaging device not possible with conventional tissue imagers.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. An imaging device for identifying subcutaneous tissue structures, the imaging device comprising:
a housing containing:
a light source configured to illuminate a subcutaneous tissue structure with substantially coherent light;
a sensor module; and
an imaging lens interposed between the subcutaneous tissue structure and the sensor module to focus an image of the subcutaneous tissue structure at the sensor module.

2. The imaging device of claim 1 and further comprising:
an identification monitor including a memory configured to store the image of the subcutaneous tissue structure.

3. The imaging device of claim 2 wherein the indicator monitor comprises a wireless transceiver configured to transmit the image to a remote station.

4. The imaging device of claim 2 wherein the identification monitor comprises:
the memory comprising a database of a plurality of record images of subcutaneous tissue structure of different individuals; and
a comparator configured to compare the stored image of subcutaneous tissue structure with the plurality of record images to uniquely identify an individual associated with the stored image based on whether the first image substantially matches one of the record images.

5. The imaging device of claim 4 wherein the comparator performs the comparison on a pixel-by-pixel basis via at least one of:
a pixel intensity parameter;
a pixel position parameter; and
a pixel volume parameter.

6. The imaging device of claim 1 and further comprising:
the housing including a contact surface for receiving placement of a first body portion of a first individual containing the subcutaneous tissue structure, the contact surface comprising a transparent member positioned to enable light from the light source to illuminate the subcutaneous tissue structure and to receive light reflected from the subcutaneous tissue structure through the transparent member to the imaging lens; and
a guiding mechanism configured to enable positioning the first body portion relative to the contact surface in a manner enabling positioning of a second body portion of a second individual in substantially the same manner as the first body portion of the first individual.

7. The imaging device of claim 6 wherein the guiding mechanism comprises a receiving member shaped and variably sized for partially enclosing a fingertip of the first body portion removably positioned over the contact surface of the housing.

8. The imaging device of claim 1 and further comprising:
a medical instrument coupled to the imaging device and, the imaging device positionable relative to the medical instrument to enable identification of a target body vessel of the subcutaneous tissue structure adjacent to the medical instrument to enable insertion of a penetrator of the medical instrument into the target body vessel; and
an identification monitor comprising:
a memory configured to store the image of the subcutaneous tissue structure; and
a comparator for comparing the stored image with a predefined criteria of the target body vessel to determine whether a vessel in the stored image substantially meets the predefined criteria.

9. The imaging device of claim 8 wherein the comparator comprises a threshold parameter defining attributes of the target body vessel, the threshold parameter including at least one of a size parameter, a position parameter, and a depth parameter.

10. The imaging device of claim 8 wherein the indicator monitor comprises an indicator mechanism configured to notify an operator whether the predefined criteria are met, the identifier mechanism comprising at least one of:
an auditory tone indicator;
a light signal indicator; and
a display indicator configured to visually display the image of the target body vessel.

11. The imaging device of claim 8 wherein the medical instrument is removably coupled to the imaging device.

12. The imaging device of claim 1 wherein the light source is configured to emit the light in a first wavelength and a second wavelength different than the first wavelength.

13. A biometric identification device comprising:
a light source configured to illuminate a subcutaneous tissue structure of a first individual with substantially coherent infrared light;
a sensor module;
an imaging lens interposed between the subcutaneous tissue structure and the sensor module to focus an image of the subcutaneous tissue structure at the sensor module; and
an image manager including a memory for storing the image associated with a first individual and a comparator for comparing the stored image with at least one record image of a subcutaneous tissue structure associated with at least one other individual to determine whether the stored image substantially matches the at least one record image.

14. The biometric identification device of claim 13 wherein the image manager is configured to evaluate an absorption coefficient over time to determine whether an oxygen content of the subcutaneous tissue structure varies over time.

15. The biometric identification device of claim 13 wherein the substantially coherent infrared light emitted by the light source comprises a first wavelength of light and a second wavelength different than the first wavelength of light and wherein the image manager is configured to identify that at least one vein of the subcutaneous tissue structure is depth dependent relative to a skin surface over the subcutaneous tissue structure.

16. A medical instrument system comprising:
an imaging device comprising:
a light source configured to illuminate a subcutaneous tissue structure with substantially coherent infrared light;
a sensor module;
an imaging lens interposed between the subcutaneous tissue structure and the sensor module to focus an image of the subcutaneous tissue structure at the sensor module; and
an image manager including a memory for storing the image and a comparator for comparing the stored image with a model image of a target body vessel to determine whether a subject body vessel in the stored image substantially matches the target body vessel in the model image; and
an insertable medical instrument coupled to imaging device,
wherein a penetrator of the medical instrument is positionable for insertion into the subject body vessel of the subcutaneous tissue structure when the predefined criteria is met.

17. The medical instrument system of claim 16 wherein imaging manager is configured to continually acquire a plurality of the stored images over time and compare each of the stored images until one of the stored images substantially matches the model image at which time the image manager alerts an operator of the substantial matching status via at least one of an visual indication and an auditory indication.

18. A method of imaging subcutaneous tissue structures, the method comprising:
directing, via a light source, substantially coherent infrared light onto a body portion to illuminate a subcutaneous tissue structure within the body portion;
focusing a first image of the subcutaneous tissue structure from beneath a skin surface onto the sensor module via an imaging lens interposed between the body portion and the sensor module; and
evaluating the first image relative to a predefined criteria to enable performing a function associated with the subcutaneous tissue structure.

19. The method of claim 18 wherein directing the substantially coherent light comprises:
arranging the light source to emit the substantially coherent infrared light as a first wavelength of infrared light and a second wavelength of infrared light different than the first wavelength of infrared light.

20. The method of claim 18 wherein evaluating the predefined criteria comprises:
arranging the predefined criteria to include at least one of:
a model image of body vessel;
at least one record image of a body vessel associated with an individual; and
at least one quantitative parameter associated with a target body vessel to enable determining whether a subject body vessel of the subcutaneous tissue structure meets the predefined criteria.
